(19) United States
(12) Patent Application Publication
(10) Pub. No.: US 2025/0295865 A1
(43) Pub. Date: Sep. 25, 2025

(54) A sub-assembly for a medicament delivery device

(71) Applicant: SHL Medical AG, Zug (CH)

(72) Inventor: Anders Karlsson, Nacka Strand (SE)

(21) Appl. No.: 18/863,412

(22) PCT Filed: Apr. 28, 2023
(86) PCT No.: PCT/EP2023/061275

§ 371 Date: Nov. 6, 2024

(30) Foreign Application Priority Data
May 12, 2022 (EP) 22173097.1

(51) Int. Cl. A61M 5/32; A61M 5/20

(52) U.S. Cl. CPC A61M 5/3245; A61M 5/20; A61M 2005/2013; A61M 2005/3254

(57) ABSTRACT

The present disclosure provides a sub-assembly for a medicament delivery device, the sub-assembly comprising a housing extending from a proximal end to a distal end along a longitudinal axis, and a medicament delivery member guard arranged telescopically inside the housing. The medicament delivery member guard is arranged to move from a proximal position relative to the housing in which the medicament delivery member guard extends from the proximal end of the housing to a distal position relative to the housing. When in the distal position, a contact portion of the medicament delivery member guard extends further from the longitudinal axis than an outer surface of the proximal portion of the housing, so that, during an injection, contact between an injection site and the outer surface of the proximal portion of the housing is limited due to contact between said injection site and the contact portion of the medicament delivery member guard. The contact portion of the medicament delivery member guard comprises a protrusion and the housing comprises a corresponding cut-out in the outer surface of the proximal portion of the housing, the protrusion being arranged so that it extends through the corresponding cut-out when the medicament delivery member guard is in the distal position relative to the housing.

### CROSS-REFERENCE TO RELATED APPLICATIONS

The present application is a U.S. National Phase Application pursuant to 35 U.S.C. § 371 of International Application No. PCT/EP2023/061275 filed Apr. 28, 2023, which claims priority to EP patent application Ser. No. 22/173,097.1 filed May 12, 2022. The entire disclosure contents of these applications are herewith incorporated by reference into the present application.

### TECHNICAL FIELD

Sub-assemblies for medicament delivery devices, and particularly sub-assemblies with a housing and a medicament delivery member guard arranged telescopically inside the housing.

### BACKGROUND

Medicament delivery devices such as autoinjectors are used for the administration of various drugs to various patient populations. The patient profile typically varies between drugs, and some drugs have patient populations with a significant number of individuals with a very high BMI (body mass index). In patients with a very high BMI, friction between the skin and the medicament delivery device can obstruct triggering of medicament delivery devices such as autoinjectors. The applicant has appreciated that it would be beneficial to take steps to ameliorate this issue.

### SUMMARY

Reference should now be made to the appended claims.

In the present disclosure, when the term “distal direction” is used, this refers to the direction pointing away from the dose delivery site (injection site) during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

An aspect concerns a sub-assembly for a medicament delivery device, the sub-assembly comprising a housing extending from a proximal end to a distal end along a longitudinal axis, and a medicament delivery member guard arranged telescopically inside the housing. The medicament delivery member guard is arranged to move from a proximal position relative to the housing in which the medicament delivery member guard extends from the proximal end of the housing to a distal position relative to the housing. When in the distal position, a contact portion of the medicament delivery member guard extends further from the longitudinal axis than an outer surface of the proximal portion of the housing, so that, during an injection, contact between an injection site and the outer surface of the proximal portion of the housing is limited due to contact between said injection site and the contact portion of the medicament delivery member guard. The contact portion of the medicament delivery member guard comprises a protrusion and the housing comprises a corresponding cut-out in the outer surface of the proximal portion of the housing, the protrusion being arranged so that it extends through the corresponding cut-out when the medicament delivery member guard is in the distal position relative to the housing. The sub-assembly could be used on various medicament delivery devices such as autoinjectors, but could be particularly useful for autoinjectors containing drugs that are given to a patient population with a higher BMI than average.

Optionally, the housing and the medicament delivery member guard are coaxial. Optionally, the housing is tubular. Optionally, the housing is an outer housing. Optionally, the contact portion of the medicament delivery member guard comprises a plurality of protrusions and the housing comprises a plurality of corresponding cut-outs. Optionally, the contact portion of the medicament delivery member guard comprises four protrusions and the housing comprises four corresponding cut-outs. Optionally, when the medicament delivery member guard is in the distal position relative to the housing, the medicament delivery member guard does not extend from the proximal end of the housing.

An aspect concerns a medicament delivery device comprising the sub-assembly of any previous claim. Optionally, the proximal portion of the housing is an outermost housing of the medicament delivery device. Optionally, the medicament delivery device is an autoinjector.

An aspect concerns a sub-assembly for a medicament delivery device, the sub-assembly comprising a tubular outer housing extending from a proximal end to a distal end along a longitudinal axis and a medicament delivery member guard arranged telescopically partly inside the outer housing, wherein the medicament delivery member guard is arranged to move from a proximal position relative to the outer housing in which the medicament delivery member guard extends from the proximal end of the outer housing to a distal position relative to the outer housing, and wherein, when in the distal position, a contact portion of the medicament delivery member guard extends further from the longitudinal axis than an outer surface of the proximal portion of the outer housing, so that, when in use, contact between the injection site and the outer surface of the proximal portion of the outer housing is reduced due to contact between the injection site and the contact portion of the medicament delivery member guard.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows part of an autoinjector.

FIG. 2 shows the autoinjector of FIG. 1 with the cap removed and with the needle guard in its proximal position relative to the housing.

FIG. 3 shows the autoinjector of FIG. 1 with the cap removed and the needle guard in its distal position relative to the housing.

FIGS. 4 and 5 show the autoinjector of FIGS. 2 and 3 respectively in context with an injection site.

### DETAILED DESCRIPTION

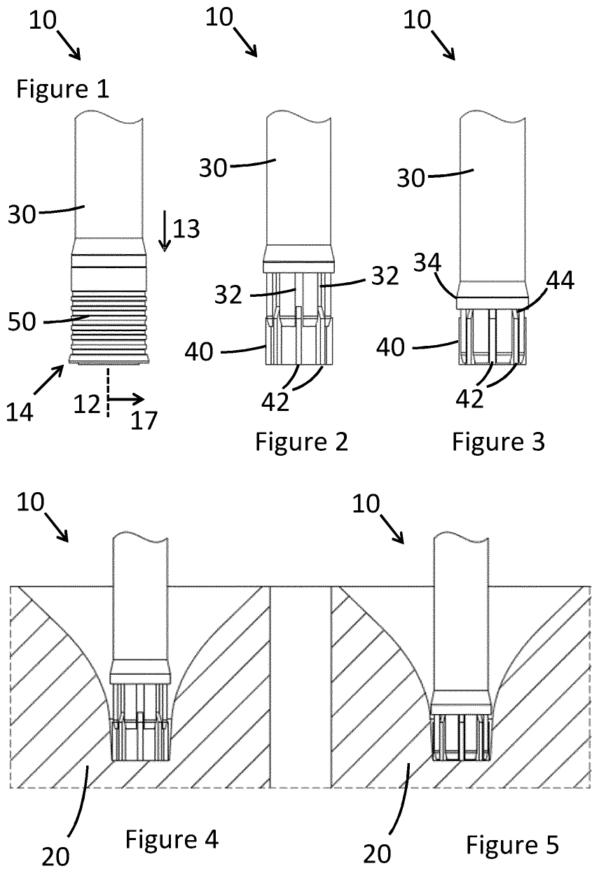

A sub-assembly for a medicament delivery device (in this example an autoinjector) will now be described with reference to FIGS. 1 to 5. In general, the sub-assembly comprises a housing 30 (in this example an outer housing) and a medicament delivery member guard (in this example a needle guard) 40. The needle guard 40 is arranged telescopically inside the housing, and the needle guard 40 is arranged to move from a proximal position relative to the housing in which the needle guard extends from the proximal end of the housing to a distal position relative to the housing. When in the distal position, a contact portion of the needle guard 40 extends further from the longitudinal axis 12 than an outer surface of the proximal portion of the housing, so that, during use, contact between an injection site 20 (see FIGS. 4 and 5) and the outer surface of the proximal portion of the housing is reduced due to contact between said injection site 20 and the contact portion of the needle guard. The contact portion of the needle guard comprises a protrusion and the housing comprises a corresponding cut-out 32 in the outer surface of the proximal portion of the housing 30, the protrusion 42 being arranged so that it extends through the corresponding cut-out 32 when the needle guard is in the distal position relative to the housing.

For reference, an axis (longitudinal axis) 12, an axial direction 13, a proximal end 14 (of the autoinjector) and a radial direction 17 are shown in FIG. 1.

FIG. 1 shows part of a medicament delivery device (in this example an autoinjector), including a housing 30 and a cap 50. The cap 50 is optional. FIG. 2 shows the autoinjector once the cap 50 has been removed. The needle guard 40 is in the proximal position; in this example, the distal end of the protrusions 42 is in the proximal end of the cut-outs 32 when the needle guard 40 is in the proximal position. Once the needle guard 40 is pushed in the distal direction relative to the housing (typically by a user holding the housing and pushing the needle guard 40 against an injection site to initiate an injection), the protrusions 42 move in the distal direction relative to the cut-outs 32, resulting in the position shown in FIG. 3. FIGS. 4 and 5 show the autoinjector in the positions shown in FIGS. 2 and 3 respectively, with an injection site 20 shown for context.

In the depicted example, the portion of the housing immediately distal from the proximal portion of the housing comprises an optional flange 34, which has a greater diameter than the proximal portion of the housing and than the portion of the housing distal to the flange 34. This flange 34 interacts with the distal end 44 of the protrusions 42 (in this example the protrusions are longitudinally extending ribs); in this example, the distal end 44 of each protrusion is tapered (with the width of the ribs narrowing in the distal direction), though this is also optional. This interaction can provide a stop, thereby limiting the distal movement of the needle guard 40.

Examples of autoinjectors in which the concepts described herein could be used are more fully described in WO2011/123024 and PCT/EP2021/078113, for example, both of which are herein incorporated by reference. A typical autoinjector would include a powerpack to drive a plunger rod. The powerpack could provide power using a power source such as a spring, a battery or compressed gas, for example. Although not essential, the powerpack would typically be locked until the device is activated for use, for example by a rotator being rotated by the needle guard 40 (or more generally the medicament delivery member guard), or by another component such as a button. A medicament delivery container carrier such as a syringe carrier could also be provided, although the medicament delivery container could be supported by one or more other components instead, such as by a housing. Although an autoinjector 10 is described, the sub-assembly could also be used in other injectors or other medicament delivery devices more generally, including non-automatic medicament delivery devices such as pen injectors.

The needle guard 40 is arranged telescopically inside the housing. Practically speaking, this means that the needle guard is as least partly inside the housing (and is optionally completely inside the housing in the distal position, with the exception of the protrusions that extend out of the housing), and is arranged so that the needle guard can move longitudinally relative to the housing. Optionally, rotational movement of the needle guard relative to the housing is restricted or stopped. For example, a protrusion such as a longitudinally extending rib could be provided on one of the housing and needle guard and a corresponding recess could be provided on the other of the housing and needle guard to allow longitudinal movement of the needle guard relative to the housing but restrict or stop rotational movement, though this is also optional and rotational movement could also be allowed.

When the needle guard is in the distal position relative to the housing, the needle guard is shown as being aligned with the housing so that the proximal end of the housing and the proximal end of the needle guard are in the same position relative to the longitudinal axis. As a result, the needle guard is, with the exception of the protrusions, entirely within the housing in the distal position. As a result, when the needle guard is in the distal position relative to the housing, the medicament delivery member guard does not extend from the proximal end of the housing. Alternatively, the needle guard could still extend from the proximal end of the housing in the longitudinal direction (with the amount extended being reduced compared to when the needle guard is in the proximal position relative to the housing).

In this application, a needle guard is referred to, as the examples would typically include a needle as a medicament delivery member. However, other medicament delivery members such as jet injectors could be used, meaning that use of the word ‘needle’ can be generalised to ‘medicament delivery member’.

References to an injection site 20 mean the volume around the point at which the injection is delivered, including the point at which the skin is pierced and the area of skin immediately around the point at which the skin is pierced, where the skin interacts with the device.

The housing 30 described herein is typically an outer housing. As a result, the housing would typically be the outermost part of the device, meaning that a user would grip the housing when using the device. However, only the proximal-most part of the housing (i.e. the proximal portion of the housing) needs to be the outermost part of the housing (to allow the protrusions to extend through the cut-outs and to interact with the injection site, as shown in FIG. 5), and the rest of the housing does not need to be an outermost housing (and could for example be covered by a medicament delivery device add-on). The proximal portion of the housing is the portion of the housing at the proximal end of the housing, which the protrusions extend through when the needle guard is in the distal position.

Various different configurations could be used for the protrusions 42 and the corresponding cut-outs 32. The cut-outs 32 and the protrusions 42 are shown as spaced out equally around the axis, though this is optional. A plurality of protrusions and cut-outs is shown, although providing only one protrusion and cut-out would already provide an effect. In a preferred example, a plurality of protrusions, for example 3 or more, 4 or more, or 5 or more protrusions, for example 4 to 8 protrusions, are provided (in a particular example 6 protrusions), along with the same number of corresponding cut-outs. The protrusions 42 would typically be longitudinally aligned with the cut-outs 32 when the needle guard is in the proximal position, though this is optional (for example, the needle guard could first be rotated into position relative to the housing, as in PCT/EP2020/078920, which is herein incorporated by reference). The protrusions are (approximately) the same length (in the longitudinal direction) as the cut-outs in the depicted example, although the protrusions could alternatively be longer or shorter than the cut-outs.

In the depicted example, the distal end of the protrusions 42 is in the proximal end of the cut-outs 32 when the needle guard 40 is in the proximal position. Alternatively, the distal end of the protrusions 42 could be proximally arranged relative to the cut-outs 32 when the needle guard 40 is in the proximal position. Optionally, there is a gap in the longitudinal direction between the distal end of the protrusions 42 and the proximal end of the cut-outs 32.

The cap 50 is shown with circumferentially extending ribs on its outer surface, which can help a user to grip the cap, but this particular design of cap is optional, and various other cap designs could be used instead.

The housing is typically tubular. The housing and the needle guard are typically coaxial. The housing 30 and the needle guard 40 are each shown as a single integral part. However, they could alternatively be made of two or more separate parts. For example, the proximal portion of the housing and the corresponding part of the needle guard could each be separate parts that can be (removably or permanently) clipped on to an existing medicament delivery device as an add-on made up of two components (the two components being a housing proximal portion and a needle guard proximal portion).

The delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-10. (canceled)

11. A sub-assembly for a medicament delivery device, the sub-assembly comprising:
a housing extending from a proximal end to a distal end along a longitudinal axis; and
a medicament delivery member guard arranged telescopically inside the housing,
wherein the medicament delivery member guard is arranged to move from a proximal position relative to the housing in which the medicament delivery member guard extends from the proximal end of the housing to a distal position relative to the housing,
wherein, when in the distal position, a contact portion of the medicament delivery member guard extends further from the longitudinal axis than an outer surface of the proximal portion of the housing, so that, during an injection, contact between an injection site and the outer surface of the proximal portion of the housing is limited due to contact between said injection site and the contact portion of the medicament delivery member guard, and
wherein the contact portion of the medicament delivery member guard comprises a protrusion and the housing comprises a corresponding cut-out in the outer surface of the proximal portion of the housing, the protrusion being arranged so that it extends through the corresponding cut-out when the medicament delivery member guard is in the distal position relative to the housing.

12. The sub-assembly of claim 11, wherein the housing and the medicament delivery member guard are coaxial.

13. The sub-assembly of claim 11, wherein the housing is tubular.

14. The sub-assembly of claim 11, wherein the housing is an outer housing.

15. The sub-assembly of claim 11, wherein the contact portion of the medicament delivery member guard comprises a plurality of protrusions and the housing comprises a plurality of corresponding cut-outs.

16. The sub-assembly of claim 11, wherein the contact portion of the medicament delivery member guard comprises four protrusions and the housing comprises four corresponding cut-outs.

17. The sub-assembly of claim 11, wherein, when the medicament delivery member guard is in the distal position relative to the housing, the medicament delivery member guard does not extend from the proximal end of the housing.

18. A medicament delivery device comprising the sub-assembly of claim 11.

19. The medicament delivery device of claim 18, wherein the proximal portion of the housing is an outermost housing of the medicament delivery device.

20. The medicament delivery device of claim 18, wherein the medicament delivery device is an autoinjector.
